SEQUENCE LISTING (12 sequences)

SEQ ID NO: 1 - DNA, 723 bp, synthetic construct
  source 1..723: mol_type = other DNA
atgaaaatgagtggctttagcatagaagaaaaggtacatgaatttgaatctaagggattccttgaaatctcaaatgaaatctttttacaagaggaagagaatcatcgtttattaacacaagcacagttagattattataatttggaagatgatgcgtacggtgaatgccgtgctggatcttattcaaggtatataaagtatgttgattcaccagattatattttagataatagtaatgattacttccaatctaaagaatataactatgatgatggcggtaaagttagacagttccatagcataaatgatagttttttatataatcctttaattcaaaatatcgtgcgtttcgatactgaatttgcatttaaaacaaatataatagatacaagtaaagatttaattataggtttacatcaagtaagatataaagctactaaagaaagaccatcttttagttcacctatttggttacataaagatgatgaaccagtagtgtttttacaccttatgaatttaagtaatacagctattggcggagataatttaatagctaattctccaagggaaattaatcagtttataagtttgaaggagcctttagaaactttagtatttggacaaaaggttttccatgccgtaacgccacttggaacagaatgtagtactgaagcttttcgtgatattttattagtaacattttcttataaggagacaaaataa

SEQ ID NO: 2 - protein, 240 aa, synthetic construct
MKMSGFSIEEKVHEFESKGFLEISNEIFLQEEENHRLLTQAQLDYYNLEDDAYGECRAGSYSRYIKYVDSPDYILDNSNDYFQSKEYNYDDGGKVRQFHSINDSFLYNPLIQNIVRFDTEFAFKTNIIDTSKDLIIGLHQVRYKATKERPSFSSPIWLHKDDEPVVFLHLMNLSNTAIGGDNLIANSPREINQFISLKEPLETLVFGQKVFHAVTPLGTECSTEAFRDILLVTFSYKETK

SEQ ID NO: 3 - DNA, 1995 bp, synthetic construct
  source 1..1995: mol_type = other DNA
atgactgaagatcacgcggtggtgcgttcggacggcggtctgtcccggaggagcttcgcggcggtggccggcacagccacggtcgccacagccctgaccagcggtgtcgccgcggccctgcccgcaccggccgcctcgggcgacagccgtggcgccgacttcgaccggtgcctggccgtcgctcgcgcactcctcgtcctggactccgacgaccgccccctcgtcccgcgttaccagagcgtcctccagaagggactgccggctcaacgacggacacggcccaagaacgtcctggtcatcggcgccggcccggccgggctggtggccgcctggttgctgaagagggcaggtcaccgggtgacggtgctggaggccaacggcaaccgagcgggcggacgcgtcaagaccttccggagcggtggccacgaacgcgccgagcagcccttcgccgatccccgtcagtacgccgaagcgggcgcgatgcgcatcccgggcagccaccccctggtgatggagctgatcgaccagttcgagctgaagaagcggcgtttccactacgtcgacgtcgacagcgagggacgtcccgccaaccgcacctggatccacgtcaacggcatccgcgtgcggcgcgccgactacgcccgcgcgccccggcgcgtgaaccggtccttcggtgtcccccgggcccactgggacacccccgccgccgccatcctgcgctccgtgctggacccggtgcgcgacgagttcagccgcgtcggccgcgacggcaagcgggtcgacaagccgctcccggagcggctgcagggctgggcccgggtggtgcagcgcttcggtgactggtcgatgttccgctttctcaccgaacacgccggcctcgacgaacggaccatcgacctgatcggcaccctggagaacctcacctcccgtctccctctgtccttcatccacagcttcatcggctcctccctcatcagccccgacacgcccttctacgagctggagggcggcacggccgtactaccggacgccctgctggagcgcgtgcgcaaggacgtgcggttcgaccgccgtgtcacgcgcatccagtaccaccaccccgaccgcccgtcgccggacgtcgagcaggtccggagcaagggcccgcacgtgtgggtggacaccgtgtcggagggccgcgacggacctgtcgtacgcgagcagttcaccgcggacgtcgccgtggtcacggtgccgttctccggactgcgccacgtacagatcgccccgcccctgtcgtacggcaagcgtcgggcggtctgcgagttgcactacgacagcgcgaccaaggtgctgctggagttcagccgccgctggtgggagttcgacgaggccgactggaaacgggagctgcgcgccgtcgacccggggctgtacgacgcctaccgcaccggccgggccgccgcggacggcagcctgctcggcgcccatccctccgtgcccgccgggcacatcacagcgggtcagcgcacccactacgccgccaaccgcgcggtcgcgcgcgaccagccggaggccgtcgacgtcgtagggggcggatcggtgtcggacaacgccaaccggttcatgttccatccctcccatccggttccgggcagcgccgggggcgtggtcctggcctcctacagctgggcggacgacgcgctgcgctgggactccctggacgacgaggcacggtacccccacgctctgtgcggcctccagcaggtctacggccagcgcatcgaggtcttctacaccggcgcgggccgcacgcagagctggttgcgcgacccctacgcctacggagaggcgtccgtgctcctgcccgggcagcacacggagttgttgtccgccatccccgtggctgaggggccgttgcacttcgccggggaccacacgtccgtcaagccggcatggatcgagggagctgtggagtccgccgtgcgtgccgccttggagatccacacagcgtga

SEQ ID NO: 4 - protein, 664 aa, synthetic construct
MTEDHAVVRSDGGLSRRSFAAVAGTATVATALTSGVAAALPAPAASGDSRGADFDRCLAVARALLVLDSDDRPLVPRYQSVLQKGLPAQRRTRPKNVLVIGAGPAGLVAAWLLKRAGHRVTVLEANGNRAGGRVKTFRSGGHERAEQPFADPRQYAEAGAMRIPGSHPLVMELIDQFELKKRRFHYVDVDSEGRPANRTWIHVNGIRVRRADYARAPRRVNRSFGVPRAHWDTPAAAILRSVLDPVRDEFSRVGRDGKRVDKPLPERLQGWARVVQRFGDWSMFRFLTEHAGLDERTIDLIGTLENLTSRLPLSFIHSFIGSSLISPDTPFYELEGGTAVLPDALLERVRKDVRFDRRVTRIQYHHPDRPSPDVEQVRSKGPHVWVDTVSEGRDGPVVREQFTADVAVVTVPFSGLRHVQIAPPLSYGKRRAVCELHYDSATKVLLEFSRRWWEFDEADWKRELRAVDPGLYDAYRTGRAAADGSLLGAHPSVPAGHITAGQRTHYAANRAVARDQPEAVDVVGGGSVSDNANRFMFHPSHPVPGSAGGVVLASYSWADDALRWDSLDDEARYPHALCGLQQVYGQRIEVFYTGAGRTQSWLRDPYAYGEASVLLPGQHTELLSAIPVAEGPLHFAGDHTSVKPAWIEGAVESAVRAALEIHTA

SEQ ID NO: 5 - DNA, 2181 bp, synthetic construct
  source 1..2181: mol_type = other DNA
atgagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtgccaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgcggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaa

SEQ ID NO: 6 - protein, 726 aa, synthetic construct
MSTSDDIHNTTATGKCPFHQGGHDQSAGAGTTTRDWWPNQLRVDLLNQHSNRSNPLGEDFDYRKEFSKLDYYGLKKDLKALLTESQPWWPADWGSYAGLFIRMAWHGAGTYRSIDGRGGAGRGQQRFAPLNSWPDNVSLDKARRLLWPIKQKYGQKISWADLFILAGNVALENSGFRTFGFGAGREDVWEPDLDVNWGDEKAWLTHRHPEALAKAPLGATEMGLIYVNPEGPDHSGEPLSAAAAIRATFGNMGMNDEETVALIAGGHTLGKTHGAGPTSNVGPDPEAAPIEEQGLGWASTYGSGVGADAITSGLEVVWTQTPTQWSNYFFENLFKYEWVQTRSPAGAIQFEAVDAPEIIPDPFDPSKKRKPTMLVTDLTLRFDPEFEKISRRFLNDPQAFNEAFARAWFKLTHRDMGPKSRYIGPEVPKEDLIWQDPLPQPIYNPTEQDIIDLKFAIADSGLSVSELVSVAWASASTFRGGDKRGGANGARLALMPQRDWDVNAAAVRALPVLEKIQKESGKASLADIIVLAGVVGVEKAASAAGLSIHVPFAPGRVDARQDQTDIEMFELLEPIADGFRNYRARLDVSTTESLLIDKAQQLTLTAPEMTALVGGMRVLGANFDGSKNGVFTDRVGVLSNDFFVNLLDMRYEWKATDESKELFEGRDRETGEVKFTASRADLVFGSNSVLRAVAEVYASSDAHEKFVKDFVAAWVKVMNLDRFDLL

SEQ ID NO: 7 - DNA, 60 bp, synthetic construct
  source 1..60: mol_type = other DNA
ctggaacagtatgcaaatcagctggcagatcagattatcaaagaagcaaccgaaggttgc

SEQ ID NO: 8 - protein, 20 aa, synthetic construct
LEQYANQLADQIIKEATEGC

SEQ ID NO: 9 - DNA, 150 bp, synthetic construct
  source 1..150: mol_type = other DNA
agcctgcgtgaatgtgaactgtatgttcagaaacataatattcaggccctgctgaaagatagcattgttcagctgtgtaccgcacgtccggaacgtccgatggcatttctgcgcgaatattttgaacgtctggaaaaagaagaagccaaa

SEQ ID NO: 10 - protein, 50 aa, synthetic construct
SLRECELYVQKHNIQALLKDSIVQLCTARPERPMAFLREYFERLEKEEAK

SEQ ID NO: 11 - DNA, 15 bp, synthetic construct
  source 1..15: mol_type = other DNA
ggtggtggtggttca

SEQ ID NO: 12 - protein, 5 aa, synthetic construct
GGGGS

(19) United States
(12) Patent Application Publication
(10) Pub. No.: US 2025/0346934 A1
(43) Pub. Date: Nov. 13, 2025

(54) SELF-ASSEMBLY ENZYME SYSTEM SUPPLYING A-KETOGLUTARATE AND APPLICATION THEREOF IN CATALYTIC SYNTHESIS OF 4-HYDROXYISOLEUCINE

(71) Applicant: Jiangnan University, Wuxi (CN)

(72) Inventors: Yao NIE, Wuxi (CN); Yan XU, Wuxi (CN); Huan LIU, Wuxi (CN)

(21) Appl. No.: 19/274,664

(22) Filed: Jul. 21, 2025

Related U.S. Application Data
(63) Continuation of application No. PCT/CN2024/116574, filed on Sep. 3, 2024.

(30) Foreign Application Priority Data
Oct. 12, 2023 (CN) 202311322018.9

(51) Int. Cl. C12P 13/06; C12N 9/02; C12N 9/06; C12N 9/08; C12N 15/70; C12R 1/19

(52) U.S. Cl. CPC C12P 13/06; C12N 9/0022; C12N 9/0065; C12N 9/0071; C12N 15/70; C12Y 104/03011; C12Y 111/01021; C12Y 114/11; C12R 2001/19

(57) ABSTRACT

Disclosed are a self-assembly enzyme system supplying α-ketoglutarate (α-KG) and application thereof in catalytic synthesis of 4-hydroxyisoleucine. In the present disclosure, glutamate oxidase catalyzes glutamate to generate α-KG, and catalase-peroxidase decomposes a byproduct H₂O₂. An interaction between RIAD and RIDD and a covalently linked combined state can mediate higher-order structures of various self-assembly enzymes. The LGOX/KatG self-assembly system is constructed through the affinity of short peptides in vitro to eliminate H₂O₂ in situ, thereby eliminating the inhibitory effect of H₂O₂ on Fe(II)/α-KG DOs, and facilitating efficient and high-yield production of 4-HIL in a one-pot cascade reaction with IDO, with a highest yield up to 95% at a substrate concentration of 100 mM.

### REFERENCE TO SEQUENCE LISTING

The instant application contains a Sequence Listing in XML format as a file named “PC250005A.xml”, created on 2025-07-10, of 18,384 byte in size, and which is hereby incorporated by reference in its entirety.

### TECHNICAL FIELD

The present disclosure belongs to the technical field of biocatalysis, and relates to a self-assembly enzyme system supplying α-ketoglutarate and application thereof in catalytic synthesis of 4-hydroxyisoleucine.

### BACKGROUND

(2S,3R,4S)-4-hydroxyisoleucine (4-HIL) is a natural non-proteinogenic amino acid, initially discovered in seeds of the herb Trigonella foenum-graecum. It exhibits potential insulinotropic bioactivity, and is considered a highly promising antidiabetic drug. Currently, there are many methods for obtaining 4-HIL, including chemical extraction, chemo-enzymatic method, biocatalytic synthesis, and the like. Compared with traditional chemical methods, which often suffer from poor selectivity and generate serious environmental pollution, the biocatalytic method offers unprecedented selectivity for reaction activation of C-H bonds, as well as high catalytic efficiency, low energy consumption, and environmental friendliness, which meets the requirements of green chemistry and is suitable for industrial applications. Owing to their remarkable biocatalytic potential, Fe(II)/α-ketoglutarate-dependent dioxygenases (Fe(II)/α-KG DOs) are ideal candidates for future chemoenzymatic synthesis and enzyme engineering for inert C-H bond activation and synthesis of important chiral pharmaceutical compounds. L-Isoleucine dioxygenase (EC 1.14.11.45, IDO) is an Fe(II)/α-KG DOs capable of stereo- and region-selectively hydroxylating various hydrophobic aliphatic L-amino acids. When L-isoleucine (L-Ile) is used as a substrate, it can be stereoselectively hydroxylated at a C4-position to generate 4-HIL.

In a reaction system where Fe(II)/α-KG DOs are used as catalysts to catalyze the activation of C-H bonds, a large amount of α-ketoglutarate (α-KG) needs to be provided as a co-substrate. Insufficient supply of α-KG will lead to low catalytic efficiency of Fe(II)/α-KG DOs. Out of consideration for economy and industrial feasibility, it is necessary to integrate efficient and low-cost α-KG regeneration systems. Therefore, researchers have focused on bioconversion method to provide α-KG for Fe(II)/α-KG DOs. Through metabolic engineering, the tricarboxylic acid (TCA) cycle of host cells can be reconstructed or optimized to enable in vivo accumulation of α-KG, and provide co-substrates for Fe(II)/α-KG DOs in vivo. However, the modification of TCA metabolic pathway often adversely affects the growth of host cells. In addition, the complexity of an in vivo environment limits the accumulation of α-KG, making it difficult to meet the catalytic demand of Fe(II)/α-KG DOs.

At present, it is industrially feasible to catalyze the inexpensive and readily available substrate L-glutamic acid by a bioenzymatic method to produce α-KG. With high substrate specificity but without the cofactor, L-glutamate oxidase (LGOX) is now regarded as a key enzyme for producing α-KG. LGOX catalyzes the conversion of L-glutamic acid into α-KG, and produces ammonia (NH₃) and hydrogen peroxide (H₂O₂) at the same time. However, the strong oxidizing property of H₂O₂ rapidly oxidizes Fe²⁺, which is required for Fe(II)/α-KG DOs reaction, which is unfavorable for the entire coupling reaction. Catalase-peroxidase can catalyze the degradation of H₂O₂, therefore, a multi-enzyme cascade system is used to eliminate H₂O₂ produced by LGOX.

For example, exogenous catalase-peroxidase is directly added/immobilized in an LGOX system, a one-pot/two-step cascade reaction is performed in vitro, and LGOX and catalase-peroxidase are co-expressed or fused in vivo for whole-cell catalysis, etc. However, a free multi-enzyme system still fails to completely prevent the accumulation of H₂O₂, which suppresses the enzyme activity and reaction of subsequent reducing environment-dependent Fe(II)/α-KG DOs, indicating the incompatibility of the reaction in the cascade process. The complexity of the reaction is significantly increased, posing challenges for its industrial application.

### SUMMARY

The technical problem to be solved by the present disclosure is that the catalysis of Fe(II)/α-KG-dependent dioxygenase family requires a large amount of α-ketoglutarate (α-KG) as a co-substrate. The current strategy for supplying a co-substrate for dioxygenase is to cascade L-glutamate oxidase (LGOX) and catalase-peroxidase (KatG) to catalyze the production of α-KG and eliminate H₂O₂ produced in the catalytic process, but the cascade system will inevitably result in the accumulation of H₂O₂, which seriously inhibits the activity of isoleucine dioxygenase (IDO).

An objective of the present disclosure is to provide a method for supplying a co-substrate α-KG to dioxygenase based on a multi-enzyme cascade system involving self-assembly enzymes. In the present disclosure, a RIAD short peptide is added to a C-terminal of LGOX using a linker peptide, and a RIDD peptide is added to a C-terminal of KatG using a linker peptide. By making use of the high-affinity and specific interaction between the RIAD and RIDD peptides, LGOX and KatG form a self-assembly enzyme system, thereby eliminating in situ a byproduct H₂O₂produced during the catalytic process of LGOX, preventing the inhibitory effect of H₂O₂ on dioxygenase activity and promoting the production of 4-HIL. Finally, a common cascade reaction system is established for converting hydroxyamino acids using dioxygenase. The self-assembly enzyme system obtained by the present disclosure can catalyze the formation of 4-IL by using L-isoleucine and L-glutamate as starting substrates and cascading the catalysis of L-isoleucine dioxygenase, LGOX via a two-step method or a one-pot method.

A first technical solution provided by the present disclosure is a self-assembly enzyme system, where the self-assembly enzyme system includes a recombinant protein LGOX-RIAD and a recombinant protein KatG-RIDD; and the recombinant protein LGOX-RIAD is LGOX with a RIAD short peptide added to a C-terminal, the recombinant protein KatG-RIDD is KatG with a RIDD short peptide added to a C-terminal, and the recombinant protein LGOX-RIAD and the recombinant protein KatG-RIDD are self-assembled through the RIAD short peptide and the RIDD short peptide.

In some embodiments, a stoichiometric ratio of the recombinant protein LGOX-RIAD to the recombinant protein KatG-RIDD is 1:2.

In some embodiments, the LGOX is linked to the RIAD short peptide using a linker (GGGGS)ₙ, where n is 4, 5 or 6.

In some embodiments, the KatG is linked to the RIDD short peptide using a linker (GGGGS)ₙ, where n is 4, 5 or 6.

In some embodiments, the LGOX is linked by (GGGGS)₄ and RIAD to obtain the recombinant protein LGOX-RIAD, and the KatG is linked by (GGGGS)₄ and RIDD to obtain the recombinant protein KatG-RIDD, and a resulting self-assembly enzyme system is designated as LK-1.

In some embodiments, the LGOX is linked by (GGGGS)₆ and RIAD to obtain the recombinant protein LGOX-RIAD, and the KatG is linked by (GGGGS)₄ and RIDD to obtain the recombinant protein KatG-RIDD, and a resulting self-assembly enzyme system is designated as LK-3.

In some embodiments, the LGOX is linked by (GGGGS)₄ and RIAD to obtain the recombinant protein LGOX-RIAD, and the KatG is linked by (GGGGS)₅ and RIDD to obtain the recombinant protein KatG-RIDD, and a resulting self-assembly enzyme system is designated as LK-4.

In some embodiments, the LGOX is linked by (GGGGS)₄ and RIAD to obtain the recombinant protein LGOX-RIAD, and the KatG is linked by (GGGGS)₆ and RIDD to obtain the recombinant protein KatG-RIDD, and a resulting self-assembly enzyme system is designated as LK-7.

In some embodiments, the LGOX is linked by (GGGGS)₅ and RIAD to obtain the recombinant protein LGOX-RIAD, and the KatG is linked by (GGGGS)₆ and RIDD to obtain the recombinant protein KatG-RIDD, and a resulting self-assembly enzyme system is designated as LK-8.

In some embodiments, a source of the LGOX includes but is not limited to Streptomyces ghanaensis.

Further, an amino acid sequence of the LGOX is shown in SEQ IDNO:4, and a nucleotide sequence of the gene encoding the LGOX is shown in SEQ ID NO:3.

In some embodiments, a source of the KatG includes but is not limited to Escherichia coli.

Further, an amino acid sequence of the KatG is shown in SEQ ID NO:6, and a nucleotide sequence of the gene encoding the KatG is shown in SEQ ID NO:5.

In some embodiments, an amino acid sequence of the RIAD is shown in SEQ ID NO:8, and a nucleotide sequence of the gene encoding the RIAD is shown in SEQ ID NO:7.

In some embodiments, an amino acid sequence of the RIDD is shown in SEQ ID NO:10, and a nucleotide sequence of the gene encoding the RIDD is shown in SEQ ID NO:9.

In some embodiments, an amino acid sequence of a basic unit (GGGGS) of the linker is shown in SEQ ID NO: 12, and a nucleotide sequence of the gene encoding the basic unit (GGGGS) of the linker is shown in SEQ ID NO:11.

In-depth research on a mechanism of protein-protein interactions has promoted the widespread application of an interaction-driven enzyme assembly. The protein-protein interactions are usually mediated by constituent peptide sequences, and enzymes fused to interaction domains can spontaneously form multi-enzyme structures with regular sizes and symmetries. Based on the interaction between affinity short peptides, enzymes are recruited to form a scaffold-free complex, minimizing an impact on the enzyme structure and activity. The amphiphilic coiled-coil RIAD specifically binds to the stable dimer RIDD to form a precisely enzyme assembly with a stoichiometric ratio of 1:2. Its strong affinity and specificity ensure effective recognition and assembly even at low concentrations.

In some embodiments, the self-assembly enzyme system is formed by mixing the recombinant protein LGOX-RIAD and the recombinant protein KatG-RIDD at a stoichiometric ratio of 1:2 to form a protein mixture, and then be subjected to form the self-assembly enzyme system.

A second technical solution provided by the present disclosure is a method for preparing the self-assembly enzyme system described in the first technical solution. The method includes the following steps:
(1) linking the gene encoding a recombinant protein LGOX-RIAD and the gene encoding a recombinant protein KatG-RIDD into expression vectors to obtain recombinant vectors, respectively;
where the recombinant protein LGOX-RIAD is LGOX with a RIAD short peptide added to a C-terminal; and the recombinant protein KatG-RIDD is KatG with a RIDD short peptide added to a C-terminal;
(2) transforming the recombinant vectors obtained in the step (1) into Escherichia coli to obtain strains expressing LGOX-RIAD and KatG-RIDD; respectively;
(3) culturing, inducing expression the transformed Escherichia coli obtain purified recombinant protein LGOX-RIAD and recombinant protein KatG-RIDD;
(4) mixing the purified protein LGOX-RIAD and KatG-RIDD to obtain the self-assembly enzyme system.

In some embodiments, in the step (1), the LGOX is linked to the RIAD short peptide using a linker (GGGGS)ₙ, where n is 4, 5 or 6.

In some embodiments, in the step (1), the KatG is linked to the RIDD short peptide using a linker (GGGGS)ₙ, where n is 4, 5 or 6.

In some embodiments, in the step (1), the expression vector includes but is not limited to pET28a.

In some embodiments, in the step (3), the Escherichia coli is Escherichia coli BL21(DE3).

In some embodiments, in the step (4), a stoichiometric ratio of the recombinant protein LGOX-RIAD to the recombinant protein KatG-RIDD is 1:2.

A third technical solution provided by the present disclosure is a method for converting 4-HIL using the multi-enzyme cascade system. The method catalyzes the production of 4-HIL through a cascade reaction using the self-assembly enzyme system described in the first technical solution and L-isoleucine dioxygenase.

In some embodiments, the method adopts a conversion system for a two-step method or a one-pot method.

In some embodiments, the conversion system for the two-step method includes a first reaction system and a second reaction system; after a reaction of the first reaction system is completed, a reaction solution is obtained by inactivating the reaction, and the reaction solution is then added to the second reaction system for further reaction;

In the first reaction system, an initial reaction mixture contains monosodium L-glutamate and the self-assembly enzyme system; where a concentration of the monosodium L-glutamate is 50-300 mM, a concentration of the LGOX in the self-assembly enzyme system is 0.1-1 mg mL⁻¹, and a concentration of the KatG in the enzyme self-assembly system is two-fold molar concentration of the LGOX; and in the second reaction system, an initial reaction mixture contains L-isoleucine, FeSO₄·7H₂O, L-ascorbic acid, and L-isoleucine dioxygenase; a concentration of the L-isoleucine is 50-300 mM, a concentration of the FeSO₄·7H₂O is 1-5 mM, a concentration of the L-ascorbic acid is 10-50 mM, and a concentration of the L-isoleucine dioxygenase is 0.1-1 mg mL⁻¹.

In the above system for the two-step method, a conversion process is divided into two stages: the first stage of the cascade catalytic reaction involves the production of the co-substrate α-KG; the monosodium L-glutamate is used as the substrate, and a self-assembly enzyme system mediated by high-affinity short peptides is used as the catalyst according to the above concentrations; where the LGOX in the self-assembly enzyme system is used to catalyze the production α-KG from the monosodium L-glutamate to α-KG, the KatG in the self-assembly enzyme system is used to eliminate in situ the byproduct H₂O₂ in the catalytic process of LGOX, and the solution after reaction is boiled for 10 minutes to inactivate the enzyme and stored for subsequent use; and the second stage of the cascade catalytic reaction involves the synthesis of 4-HIL, where the solution after reaction obtained from the first stage is used as a starting reaction solution to provide the co-substrate α-KG, and L-isoleucine, FeSO₄·7H₂O, L-ascorbic acid, and L-isoleucine dioxygenase are added to carry out the conversion reaction according to the aforesaid concentrations.

A fourth technical solution provided by the present disclosure is application of the self-assembly enzyme system according to the first technical solution, the method according to the second technical solution, or the method according to the third technical solution in the production of 4-HIL. In some embodiments, a temperature of the conversion system for the two-step method is 25-35° C., a pH is 7.0-8.0, a conversion in the first stage lasts for 2-9 hours, a conversion in the second stage lasts for 2-9 hours, and a rotation speed is 200-400 rpm.

In some embodiments, the conversion system for the two-step method is to add the substrate and enzyme to a Tris-HCl solution with a pH of 7.0-8.0 for conversion reaction.

In some embodiments, the method adopts the one-pot conversion system. The “one-pot method” cascade catalysis refers to the simultaneous regeneration of α-KG and hydroxylation of amino acids in a single conversion system, where the conversion system is formed by monosodium L-glutamate, L-isoleucine, FeSO₄·7H₂O, L-ascorbic acid, the self-assembly enzyme system, and L-isoleucine dioxygenase.

In the conversion system for the one-pot method, the initial reaction mixture contains monosodium L-glutamate, L-isoleucine, FeSO₄·7H₂O, L-ascorbic acid, L-isoleucine dioxygenase, and the self-assembly enzyme system; where a concentration of the monosodium L-glutamate is 50-300 mM, a concentration of the L-isoleucine is 50-300 mM, a concentration of the FeSO₄·7H₂O is 1-5 mM, a concentration of the L-ascorbic acid is 10-50 mM, and a concentration of the L-isoleucine dioxygenase is 0.1-1 mg mL⁻¹; and a concentration of LGOX in the self-assembly enzyme system is 0.1-1 mg mL⁻¹, and a corresponding concentration of the KatG is two-fold molar concentration of the LGOX.

In some embodiments, the conversion system for the one-pot method is to add the substrate and enzyme to a Tris-HCl solution with a pH of 7.0-8.0 for conversion reaction.

In some embodiments, a temperature of the conversion system for the one-pot method is 25-35° C., a pH is 7.0-8.0, a conversion lasts for 2-9 hours, and a rotation speed is 200-400 rpm.

In some embodiments, a buffer system for the self-assembly enzyme system further includes a buffer solution containing Tris-HCl, EDTA, and Tween-20, where a pH of the Tris-HCl is 7.0; a concentration of the EDTA is 1 mM; and a mass concentration of the Tween-20 is 0.02%; and the buffer system is used to stabilize the proteins.

In some embodiments, the self-assembly enzyme system is any one or more of the above LK-1, LK-3, LK-4, LK-7, or LK-8.

Compared with the Prior Art, the Present Disclosure has the Following Beneficial Effects:

In the present disclosure, a RIAD short peptide is added to a C-terminal of LGOX using a linker peptide, and a RIDD peptide is added to a C-terminal of KatG using a linker peptide. By making use of the high-affinity and specific interaction between the RIAD and RIDD peptides, LGOX and KatG are self-assembled to form a self-assembly enzyme system, thereby eliminating in situ a byproduct H₂O₂ produced during the catalytic process of LGOX, preventing the inhibitory effect of H₂O₂ on dioxygenase activity and promoting the production of 4-HIL.

The self-assembly enzyme system obtained by the present disclosure can catalyze the formation of 4-HIL by using L-isoleucine and L-glutamate as starting substrates and cascading the catalysis of L-isoleucine dioxygenase, LGOX via a two-step method or a one-pot method. Using the one-pot method to prepare4-HIL, a conversion rate greater than 95% is achieved within 7 hours at an L-isoleucine concentration of 100 mM, with a space-time yield of 2 g L⁻¹ h⁻¹.

The method of the present disclosure is environmentally friendly, economical and simple.

### BRIEF DESCRIPTION OF THE DRAWINGS

FIG. 1 shows SDS-PAGE electrophoresis results after protein purification in a multi-enzyme cascade reaction system; specifically, purified LGOX (lane 1), LGOXRA (GGGGS)₄ (lane 2), LGOXRA (GGGGS)₅(lane 3), and LGOXRA (GGGGS)₆ (lane 4); and M: protein marker.

FIG. 2 shows SDS-PAGE electrophoresis results after protein purification in a multi-enzyme cascade reaction system; specifically, purified KatG (lane 5), KatGRD (GGGGS)₄ (lane 6), KatGRD (GGGGS)₅(lane 7), KatGRD (GGGGS)₆ (lane 8), and IDO (lane 9); and M: protein marker.

FIG. 3 shows KatG-LGOX tri-enzyme unit analyzed by non-reducing SDS-PAGE; specifically, LK, LK-1, LK-2, LK-3, LK-4, LK-5, LK-6, LK-7, LK-8, LK-9, and M: protein marker.

FIG. 4 shows particle sizes of assembly enzyme systems of various combinations analyzed by dynamic light scattering (DLS).

FIG. 5A shows a cascade strategy design for producing 4-HIL using a two-step method.

FIG. 5B shows a cascade strategy design for producing 4-HIL using a one-pot method.

FIG. 5C shows changes in concentrations of L-Ile, α-KG, SA, and 4-HIL when 4-IL is produced by a two-step method.

FIG. 5D shows changes in concentrations of L-Ile, α-KG, SA, and 4-HIL when 4-HIL is produced by a one-pot method.

FIG. 6 shows a route of synthesizing 4-HIL using a one-pot method based on a multi-enzyme cascade reaction system involving a self-assembly enzyme system.

FIG. 7 shows SDS-PAGE analysis results in Comparative Example 1; where Escherichia coli BL21(DE3) containing pET28a (lane 1), a supernatant of LGOX-RIAD (lane 2), a supernatant of LGOX-RIDD (lane 3), and a precipitate of LGOX-RIDD (lane 4); and M: protein marker.

### DETAILED DESCRIPTIONS OF THE EMBODIMENTS

In order to make the objectives, technical solutions and advantages of the present disclosure clearer, the present disclosure will be further described in detail below in conjunction with the specific implementations with reference to the accompanying drawings.

The following examples detected various substances in the reaction liquid phase using high-performance liquid chromatography (HPLC), including the detection of amino acids and organic acids. The detection method is as follows:

Determination of amino acid content: a sample was subjected to derivatization reaction with a fluorenylmethoxycarbonyl chloride (Fmoc-Cl) reagent, a supernatant after centrifugation was diluted to an appropriate concentration, 250 μL of the supernatant was taken, 250 μL of 0.2 mM boric acid buffer (pH 9.2) was added to the supernatant, 500 μL of 10 mM Fmoc-Cl was then added, and shaken well and reacted in a 25° C. metal bath for 10 minutes, 500 μL of 40 mM 1-aminoadamantane was added to terminate the reaction, filtering was performed using a 0.22 m organic filter for liquid chromatography analysis. Detection conditions: a Diomansil C18 column was used, with a ultraviolet detection wavelength of 263 nm, a temperature of 25° C., a flow rate of 1 mL·min⁻¹, and a sample injection volume of 10 μL. Mobile Phase A: 50 mM NaAc-HAc buffer (pH 4.2)/acetonitrile at a volume ratio of 90:10; Mobile Phase B: 50 mM NaAc-HAc buffer (pH 4.2)/acetonitrile at a volume ratio of 20:80, and Mobile Phase A and B were used in a gradient elution program, with a flow rate of 1.0 mL/minute.

Determination of organic acid content: a supernatant was taken, centrifuged and diluted to an appropriate concentration, and then filtered through a 0.22 m aqueous filter for detection. A Waters T3 column was used, with an ultraviolet absorption wavelength of 210 nm, a column temperature of 40° C., a mobile phase of 20 mM KH₂PO₄ solution, pH 2.8, a flow rate of 0.8 mL·min⁻¹, and an injection volume of 10 μL.

Methods for enzyme activity assay in the following examples:

L-Isoleucine dioxygenase enzyme activity assay method: The activity of IDO was determined according to the amount of 4-HIL produced. An IDO enzyme activity assay solution (50 mM Tris-HCl, pH 7.0) containing 10 mM Ile, 0.5 mM FeSO₄·7H₂0, 10 mM ascorbic acid, and 10 mM α-ketoglutaric acid with a final volume of 300 μL (with an IDO concentration of 0.1 g L⁻¹) was incubated at 30° C. in an incubator (Eppendorf; Hamburg, Germany) at 800 rpm for 10 minutes, and 4-HIL content in the system was then measured. An amount of enzyme that catalyzed the generation of 1 μmol of 4-HIL per minute was defined as one unit of IDO enzyme activity (U).

L-glutamate oxidase (LGOX) activity assay method: The activity of LGOX or LGOX-RIAD was determined according to the amount of α-ketoglutarate (α-KG) produced. An LGOX or LGOX-peptidase activity assay solution (50 mM Tris-HCl, pH 7.0) containing 10 mM L-Glu, and 1 mg L⁻¹ commercial catalase-peroxidase (KatG) with a final volume of 300 μL (with an LGOX concentration of 0.1 g L⁻¹) was incubated at 30° C. at 800 rpm for 10 minutes, and a content of α-ketoglutaric acid was then measured. The amount of enzyme used to release 1 μmol of α-KG per minute was defined as one unit of LGOX or LGOX-RIAD enzyme activity (U).

KatG activity assay method: The activity of KatG or KatG-RIDD was determined according to the consumption of H₂02. An enzyme activity assay solution 50 mM Tris-HCl, pH 7.0) containing 20 mM H₂O₂ with a final volume of 3 mL (with a KatG concentration of 0.1 g L⁻¹) was incubated at 30° C. at 200 rpm for 10 minutes, and an amount of reduction of H₂O₂ was detected using a UV spectrophotometer at 210 nm. The amount of enzyme used to decompose 1 mol of H₂O₂ per minute was defined as one unit of KatG or KatG-RIDD enzyme activity (U).

Strain Escherichia coli BL21(DE3) and plasmid pET28a were commercially available strains and plasmids.

The present disclosure will be further described below in conjunction with specific examples.

### Example 1: This Example Illustrates the Expression and Purification Method of LGOX-RIAD and KatG-RIDD Fusion Proteins

1. Construction of Escherichia coli BL21(DE3)/pET-28α-LGOX(pET-28a-LGOX-RIAD)

The LGOX and LGOX-linker-RIAD fusion protein fusion gene coding sequences containing enzyme restriction sites NcoI and XhoI were synthesized by a biotechnology company. Specifically, LGOX was the LGOX derived from Streptomyces ghanaensis (ATCC14672), and LGOX-RIAD was a fusion protein LGOX-linker-RIAD, that is, a linker was used to link the RIAD short peptide at a C-terminal of LGOX, and the linker was a flexible linker (GGGGS)₄-(GGGGS)₆ with different lengths. A nucleotide sequence of the gene encoding the LGOX was shown in SEQ ID NO:3, a nucleotide sequence of the gene encoding the short peptide RIAD was shown in SEQ ID NO:7, and a nucleotide sequence of the gene encoding a basic unit (GGGGS) of the linker was shown in SEQ ID NO:11. The synthesized gene fragments and the vector plasmid pET-28a were double-digested, and the synthesized gene fragments were ligated to a pET-28a plasmid using T4 ligase to obtain pET-28a-LGOX (pET-28a-LGOX-RIAD) expression plasmids with LGOX and linker lengths of (GGGGS)₄-(GGGGS)₆, respectively. The expression plasmids were then converted into an expression strain Escherichia coli BL21(DE3) to obtain recombinant Escherichia coli strains Escherichia coli BL21(DE3)/pET-28a-LGOX(pET-28a-LGOX-RIAD) expressing LGOX and LGOX-RIAD fusion proteins with different lengths of linker, that is, LGOX-RIAD (GGGGS)₃, LGOX-RIAD (GGGGS)₄, LGOX-RIAD (GGGGS)₅, and LGOX-RIAD (GGGGS)₆.

The free enzyme LGOX without connecting a short peptide and a linker was prepared in the same method using the nucleotide sequence of LGOX as shown in SEQ ID NO:3 as a control.

2. Construction of Escherichia coli BL21(DE3)/pET-28a-KatG(pET-28a-KatG-RIDD)

The KatG and KatG-linker-RIDD fusion protein fusion gene coding sequences containing enzyme restriction sites NcoI and XhoI were synthesized by a biotechnology company. Specifically, KatG was a catalase derived from Escherichia coli (W3110), and KatG-RIDD was a fusion protein KatG-linker-RIDD, that is, a linker was used to link the RIDD short peptide at a C-terminal of KatG, and the linker was a flexible linker of different lengths (GGGGS)₄-(GGGGS)₆. A nucleotide sequence of the gene encoding the KatG was shown in SEQ ID NO:5, a nucleotide sequence of the gene encoding the short peptide RIDD was shown in SEQ ID NO:9, and a nucleotide sequence of the gene encoding a basic unit (GGGGS) of the linker was shown in SEQ ID NO:11. The synthesized gene fragments and the vector plasmid pET-28a were double-digested, and the synthesized gene fragments were ligated to a pET-28a plasmid using T4 ligase to obtain pET-28a-KatG(pET-28a-KatG-RIDD) expression plasmids with KatG and linker lengths of (GGGGS)₄-(GGGGS)₆, respectively. The expression plasmids were then converted into an expression strain Escherichia coli BL21(DE3) to obtain recombinant Escherichia coli strains Escherichia coli BL21(DE3)/pET-28a-KatG(pET-28a-KatG-RIDD) expressing KatG and KatG-RIDD fusion proteins with different lengths of linker, that is, KatG-RIDD (GGGGS)₃, KatG-RIDD (GGGGS)₄, KatG-RIDD (GGGGS)ₛ, and KatG-RIDD (GGGGS)₆.

The free enzyme LGOX without connecting a short peptide and a linker was prepared in the same method using the nucleotide sequence of KatG as shown in SEQ ID NO:3 as a control.

### 3. Expression and Purification Method of LGOX-RIAD Fusion Protein

A method for preparing the recombinant pure protein enzyme was as follows:

(1) A single colony of Escherichia coli BL21(DE3)/pET-28a-LGOX-RIAD was picked from a plate with kanamycin and cultured continuously in 50 mL and 800 mL of LB medium (kanamycin 50 μg·mL⁻¹). When OD₆₀₀ reached 0.6-0.8, isopropyl-o-D-thiogalactoside was added to a final concentration of 0.5 mM, and the protein expression was induced in a constant temperature shaker at 17° C. for 12-14 hours. A cultured bacterial solution was centrifuged at 4,600×g for 10 minutes, a bacterial pellet was washed twice with 0.9% saline, and a wet bacterial pellet was then collected.

(2) The cell pellet was resuspended in a 20 mM Tris-HCl buffer (pH 7.5) containing 4 mM of 2-mercaptoethanol at a concentration of 0.1 mg L⁻¹, the resuspended cell pellet was then lysed by ultrasound in an ice-water bath, with working conditions as follows: power 40%, 2 seconds on, 3 seconds off, for 20 minutes. A supernatant was collected by centrifugation at 15,000×g for 30 minutes, the supernatant was filtered through a 0.22 m filter and slowly injected into a His-trap nickel column (GE Healthcare, Little Chalfont, UK) using an AKTA AVANT 25 instrument for gradient elution to purify the target protein. An elution buffer for washing the impurity proteins contained 200 mM imidazole, 20 mM Tris, 300 mM NaCl, pH 7.5; and an elution buffer for washing the target protein contained 600 mM imidazole, 20 mM Tris, 300 mM NaCl, pH 7.5. The purified protein was then concentrated using an Amicon® Ultra-15 centrifugal filter.

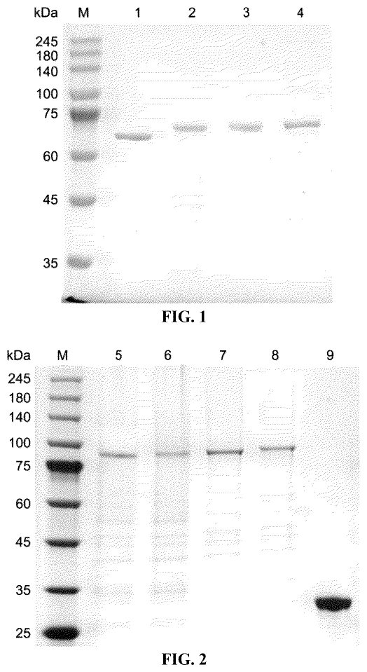

The purified protein was analyzed by SDS-PAGE, resulting bands were consistent with the theoretical molecular weight. The results are shown in FIG. 1.

### 4. Expression and Purification Method of KatG-RIDD Fusion Protein

(1) A single colony of recombinant Escherichia coli BL21(DE3)/pET-28a-KatG-RIDD was picked from a plate with kanamycin and cultured continuously in 50 mL and 800 mL of LB medium (kanamycin 50 μg·mL⁻¹). When OD₆₀₀ reached 0.6-0.8, isopropyl-o-D-thiogalactoside was added to a final concentration of 0.5 mM, and the protein expression was induced in a constant temperature shaker of 17-20° C. for 12-14 hours. A cultured bacterial solution was centrifuged at 4,600×g for 10 minutes, a bacterial pellet was washed twice with 0.9% saline, and a wet bacterial pellet was then collected.

(2) The cell pellet was resuspended in a 20 mM Tris-HCl buffer (pH 7.5) containing 4 mM of 2-mercaptoethanol at a concentration of 0.1 mg L⁻¹, the resuspended cell pellet was then lysed by ultrasound in an ice-water bath, with working conditions as follows: power 40%, 2 seconds on, 3 seconds off, for 20 minutes. A supernatant was collected by centrifugation at 15,000×g for 30 minutes, and the target protein was purified by gradient elution using a His-trap nickel column (GE Healthcare, Little Chalfont, UK) and AKTA AVANT 25. An elution buffer for washing the impurity proteins contained 80 mM imidazole, 20 mM Tris, 300 mM NaCl, pH 7.5; and an elution buffer for washing the target protein contained 300 mM imidazole, 20 mM Tris, 300 mM NaCl, pH 7.5. The purified protein was then concentrated using an Amicon® Ultra-15 centrifugal filter. The purified protein was analyzed by SDS-PAGE, resulting bands were consistent with the theoretical molecular weight. The results are shown in FIG. 2.

### Example 2: This Example Illustrates the Expression and Purification Method of IDO Protein

### 1. Construction of Escherichia Coli BL21(DE3)/pET-28a-IDO

IDO fusion protein fusion gene coding sequences containing enzyme restriction sites NcoI and XhoI were synthesized by a biotechnology company. Specifically, an amino acid sequence of L-isoleucine dioxygenase (IDO) was shown in SEQ ID NO:2, a nucleotide sequence encoding the IDO was shown in SEQ ID NO:1, the synthesized gene fragments and the vector plasmid pET-28a were double-digested, and the synthesized gene fragments were ligated to a pET-28a plasmid using T4 ligase to obtain expression plasmids pET28a-IDO, the expression plasmids were then converted into an expression strain E. coli BL21(DE3) to recombinant Escherichia coli strains Escherichia coli BL21(DE3)/pET-28a-IDO.

### 2. Expression and Purification Method of IDO Protein

A method for preparing the recombinant pure protein enzyme was as follows:

The cell pellet was resuspended in a 20 mM Tris-HCl buffer (pH 7.5) at a concentration of 0.1 mg L⁻¹, the resuspended cell pellet was then lysed by ultrasound in an ice-water bath, with working conditions as follows: power 40%, 2 seconds on, 3 seconds off, for 20 minutes. A supernatant was collected by centrifugation at 15,000×g for 30 minutes, and the target protein was purified by gradient elution using a His-trap nickel column (GE Healthcare, Little Chalfont, UK) and AKTA AVANT 25. An elution buffer for washing the impurity proteins contained 60 mM imidazole, 20 mM Tris, 280 mM NaCl, pH 7.5; and an elution buffer for washing the target protein contained 300 mM imidazole, 20 mM Tris, 300 mM NaCl, pH 7.5. The purified protein was then concentrated using an Amicon® Ultra-15 centrifugal filter and to obtain IDO. The purified protein was analyzed by SDS-PAGE, resulting bands were consistent with the theoretical molecular weight. The results are shown in FIG. 2.

Results of enzyme activity test of all enzymes in Example 1-2 are shown in Table 1.

TABLE 1
Enzyme activity results

| | Enzyme activity |
|---|---|
| Enzyme | (U · mg⁻¹) |
| IDO | 3.19 ± 0.11 |
| LGOX | 1.90 ± 0.09 |
| LGOX-RIAD (GGGGS)₃ | 1.55 ± 0.01 |
| LGOX-RIAD (GGGGS)₄ | 1.78 ± 0.10 |
| LGOX-RIAD (GGGGS)₅ | 1.64 ± 0.18 |
| LGOX-RIAD (GGGGS)₆ | 1.56 ± 0.07 |
| KatG | 5423.66 ± 100.15 |
| KatG-RIDD (GGGGS)₃ | 4646.84 ± 670.48 |
| KatG-RIDD (GGGGS)₄ | 4258.44 ± 384.63 |
| KatG-RIDD (GGGGS)₅ | 4866.78 ± 223.85 |
| KatG-RIDD (GGGGS)₆ | 4703.00 ± 176.26 |

As shown in Table 1, the enzyme activities of LGOX and KatG after connecting short peptides reached more than 78% of the wild-type enzyme activity.

### Example 3: Preparation of Self-Assembly Enzyme System

In this example, different self-assembly enzyme systems were prepared. In order to explore an optimal linker length, the assembly efficiency of LGOX-RIAD and KatG-RIDD fused with different linker lengths was analyzed.

The self-assembly enzyme systems were prepared as follows: The LGOX-RIAD and KatG-RIDD fusion enzymes with different linker lengths of fused short peptides prepared according to the method in Example 1 were mixed at a molar ratio of 1:2 to prepare a protein solution (that is, a multi-enzyme complex mixed solution), where a concentration of LGOX-RIAD was 1 mg·mL⁻¹. Using the strong specific affinity between the RIAD-RIDD short peptides, the LGOX-RIAD and KatG-RIDD in the mixed solution achieved the self-assembly of LGOX and catalase, forming the self-assembly enzyme system.

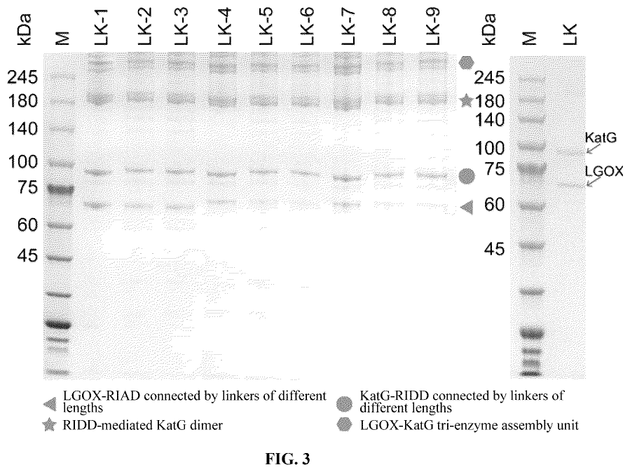

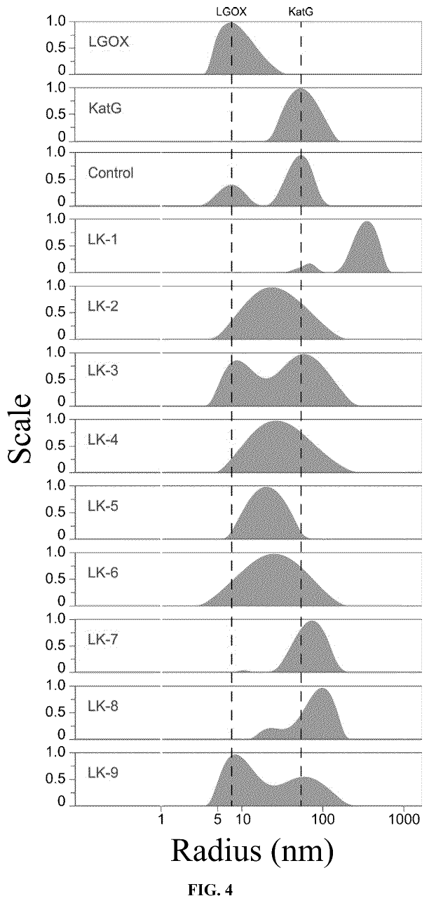

As shown in Table 2, nine different combinations of the self-assembly enzyme systems were obtained, which was used as experimental groups. A mixed solution (LK) of free enzymes LGOX and KatG was used as a control group, and other conditions were the same as those of the experimental groups. The assembly status of enzyme complexes with different linker lengths of fused short peptides was analyzed using non-reducing SDS-PAGE (FIG. 3) and dynamic light scattering (FIG. 4). Non-reducing SDS-PAGE of the nine combinations all obtained bands of an expected molecular size of about 250 KDa for the tri-enzyme assembly. In LK-1, a peak with a significantly increased radius was observed, with an average radius distribution of 345 nm. Samples LK-7 and LK-8 also showed clear peaks, with an average radius of about 80 nm. A common feature of LK-1, LK-7, and LK-8 was that the corresponding radius of LGOX was almost not distributed, indicating that all LGOX molecules were in an assembled state. Particle size distribution of LK-2, LK-4, LK-5, and LK-6 was uniform, ranging from 5 nm to 250 nm. In LK-3 and LK-9, two distinct overlapping peaks were observed, corresponding to average particle size distribution of about 10.00 nm and 55.75 nm. The results indicated that successful protein complexes were formed in all nine combinations, of which LK-1, LK-7, and LK-8 achieved complete assembly, LK-2, LK-4, LK-5, and LK-6 achieved partial assembly, and LK-3 and LK-9 had a lowest assembly level.

TABLE 2
Different self-assembly enzyme systems
obtained from nine combination methods

LGOX-RIAD

| KatG-RIDD | (GGGGS)₄ | (GGGGS)₅ | (GGGGS)₆ |
|---|---|---|---|
| (GGGGS)₄ | LK-1 | LK-2 | LK-3 |
| (GGGGS)₅ | LK-4 | LK-5 | LK-6 |
| (GGGGS)₆ | LK-7 | LK-8 | LK-9 |

### Example 4 Application of Different Self-Assembly Enzyme Systems in Catalytic Generation of α-KG

This example analyzes the reaction efficiency of assembled enzyme (that is, self-assembly enzyme system) of LGOX-RIAD and KatG-RIDD fused with different linker lengths.

In order to further explore the potential of each combination in producingα-KG, the multi-enzyme complex mixed solutions from the nine combinations prepared in Example 3 were subjected to catalytic reaction for the production of α-KG. The catalytic efficiency of the assembled enzymes was evaluated, and the mixed solution (LK) of free enzymes was used as a control. The results are shown in Table 3.

Specifically, a reaction system was 0.1 mg mL⁻¹ of LGOX (or LGOX-RIAD) and the corresponding two-fold molar amount of KatG (or KatG-RIDD), 50-300 mM (50, 100, 150, 200, 300 mM) of substrate monosodium L-glutamateand 50 mM of Tris-HCl buffer (pH 7.0), and the conversion was carried out at 200 rpm and 30° C. for 12 hours.

(1) The LK prepared in Example 3 was used to produce α-KG. When a substrate concentration was 50 mM, an accumulated amount of α-KG after 12 hours of conversion was 18.23 mM, with a molar yield of 36.47%; when a substrate concentration was 100 mM, an accumulated amount of α-KG after 12 hours of conversion was 43.45 mM, with a molar yield of 43.45%; when a substrate concentration was 150 mM, an accumulated amount of α-KG after 12 hours of conversion was 13.09 mM, with a molar yield of 8.72%; when a substrate concentration was 200 mM, an accumulated amount of α-KG after 12 hours of conversion was 17.10 mM, with a molar yield of 8.55%; when a substrate concentration was 300 mM, an accumulated amount of α-KG after 12 hours of conversion was 17.95 mM, with a molar yield of 5.99%;

(2) The LK-1 prepared in Example 3 was used to produce α-KG. When a substrate concentration was 50 mM, an accumulated amount of α-KG after 12 hours of conversion was 47.24 mM, with a molar yield of 94.48%; when a substrate concentration was 100 mM, an accumulated amount of α-KG after 12 hours of conversion was 99.24 mM, with a molar yield of 99.24%; when a substrate concentration was 150 mM, an accumulated amount of α-KG after 12 hours of conversion was 83.03 mM, with a molar yield of 55.35%; when a substrate concentration was 200 mM, an accumulated amount of α-KG after 12 hours of conversion was 70.20 mM, with a molar yield of 35.10%; when a substrate concentration was 300 mM, an accumulated amount of α-KG after 12 hours of conversion was 53.62 mM, with a molar yield of 17.87%;

(3) The LK-2 prepared in Example 3 was used to produce α-KG. When a substrate concentration was 50 mM, an accumulated amount of α-KG after 12 hours of conversion was 3.00 mM, with a molar yield of 6.00%; when a substrate concentration was 100 mM, an accumulated amount of α-KG after 12 hours of conversion was 3.61 mM, with a molar yield of 36.07%; when a substrate concentration was 150 mM, an accumulated amount of α-KG after 12 hours of conversion was 6.44 mM, with a molar yield of 4.30%; when a substrate concentration was 200 mM, an accumulated amount of α-KG after 12 hours of conversion was 0.88 mM, with a molar yield of 0.44%; when a substrate concentration was 300 mM, an accumulated amount of α-KG after 12 hours of conversion was 8.39 mM, with a molar yield of 2.80%;

(4) The LK-3 prepared in Example 3 was used to produce α-KG. When a substrate concentration was 50 mM, an accumulated amount of α-KG after 12 hours of conversion was 35.64 mM, with a molar yield of 48.62%; when a substrate concentration was 100 mM, an accumulated amount of α-KG after 12 hours of conversion was 21.48 mM, with a molar yield of 21.48%; when a substrate concentration was 150 mM, an accumulated amount of α-KG after 12 hours of conversion was 16.14 mM, with a molar yield of 10.76%; when a substrate concentration was 200 mM, an accumulated amount of α-KG after 12 hours of conversion was 20.39 mM, with a molar yield of 10.20%; when a substrate concentration was 300 mM, an accumulated amount of α-KG after 12 hours of conversion was 43.54 mM, with a molar yield of 13.51%;

(5) The LK-4 prepared in Example 3 was used to produce α-KG. When a substrate concentration was 50 mM, an accumulated amount of α-KG after 12 hours of conversion was 35.64 mM, with a molar yield of 71.28%; when a substrate concentration was 100 mM, an accumulated amount of α-KG after 12 hours of conversion was 19.47 mM, with a molar yield of 19.47%; when a substrate concentration was 150 mM, an accumulated amount of α-KG after 12 hours of conversion was 23.94 mM, with a molar yield of 15.96%; when a substrate concentration was 200 mM, an accumulated amount of α-KG after 12 hours of conversion was 22.46 mM, with a molar yield of 11.23%; when a substrate concentration was 300 mM, an accumulated amount of α-KG after 12 hours of conversion was 27.95 mM, with a molar yield of 9.32%;

(6) The LK-5 prepared in Example 3 was used to produce α-KG. When a substrate concentration was 50 mM, an accumulated amount of α-KG after 12 hours of conversion was 2.38 mM, with a molar yield of 4.77%; when a substrate concentration was 100 mM, an accumulated amount of α-KG after 12 hours of conversion was 6.21 mM, with a molar yield of 6.21%; when a substrate concentration was 150 mM, an accumulated amount of α-KG after 12 hours of conversion was 3.17 mM, with a molar yield of 2.11%; when a substrate concentration was 200 mM, an accumulated amount of α-KG after 12 hours of conversion was 8.37 mM, with a molar yield of 4.19%; when a substrate concentration was 300 mM, an accumulated amount of α-KG after 12 hours of conversion was 4.92 mM, with a molar yield of 1.64%;

(7) The LK-6 prepared in Example 3 was used to produce α-KG. When a substrate concentration was 50 mM, an accumulated amount of α-KG after 12 hours of conversion was 1.40 mM, with a molar yield of 2.81%; when a substrate concentration was 100 mM, an accumulated amount of α-KG after 12 hours of conversion was 12.02 mM, with a molar yield of 12.02%; when a substrate concentration was 150 mM, an accumulated amount of α-KG after 12 hours of conversion was 13.09 mM, with a molar yield of 8.73%; when a substrate concentration was 200 mM, an accumulated amount of α-KG after 12 hours of conversion was 4.66 mM, with a molar yield of 2.33%; when a substrate concentration was 300 mM, an accumulated amount of α-KG after 12 hours of conversion was 7.40 mM, with a molar yield of 2.47%;

(8) The LK-7 prepared in Example 3 was used to produce α-KG. When a substrate concentration was 50 mM, an accumulated amount of α-KG after 12 hours of conversion was 13.73 mM, with a molar yield of 27.36%; when a substrate concentration was 100 mM, an accumulated amount of α-KG after 12 hours of conversion was 18.10 mM, with a molar yield of 18.10%; when a substrate concentration was 150 mM, an accumulated amount of α-KG after 12 hours of conversion was 17.18 mM, with a molar yield of 11.45%; when a substrate concentration was 200 mM, an accumulated amount of α-KG after 12 hours of conversion was 12.87 mM, with a molar yield of 6.44%; when a substrate concentration was 300 mM, an accumulated amount of α-KG after 12 hours of conversion was 21.09 mM, with a molar yield of 7.03%;

(9) The LK-8 prepared in Example 3 was used to produce α-KG. When a substrate concentration was 50 mM, an accumulated amount of α-KG after 12 hours of conversion was 14.22 mM, with a molar yield of 28.45%; when a substrate concentration was 100 mM, an accumulated amount of α-KG after 12 hours of conversion was 22.39 mM, with a molar yield of 22.39%; when a substrate concentration was 150 mM, an accumulated amount of α-KG after 12 hours of conversion was 17.17 mM, with a molar yield of 11.45%; when a substrate concentration was 200 mM, an accumulated amount of α-KG after 12 hours of conversion was 15.87 mM, with a molar yield of 7.94%; when a substrate concentration was 300 mM, an accumulated amount of α-KG after 12 hours of conversion was 13.94 mM, with a molar yield of 4.65%;

(10) The LK-9 prepared in Example 3 was used to produce α-KG. When a substrate concentration was 50 mM, an accumulated amount of α-KG after 12 hours of conversion was 10.23 mM, with a molar yield of 2.46%; when a substrate concentration was 100 mM, an accumulated amount of α-KG after 12 hours of conversion was 13.57 mM, with a molar yield of 13.57%; when a substrate concentration was 150 mM, an accumulated amount of α-KG after 12 hours of conversion was 15.23 mM, with a molar yield of 10.15%; when a substrate concentration was 200 mM, an accumulated amount of α-KG after 12 hours of conversion was 14.96 mM, with a molar yield of 7.48%; when a substrate concentration was 300 mM, an accumulated amount of ax-KG after 12 hours of conversion was 4.17 mM, with a molar yield of 1.390%; and

LK-1 exhibited strong catalytic activity at all substrate concentrations. When the substrate concentrations were 50 mM and 100 mM, LK-1 achieved a yield of >800 within 6 hours, and >90% after 12 hours. In addition, LK-3 and LK-4 showed superior catalytic efficiency over other combinations and the control group across all substrate concentration gradients. The catalytic efficiency of LK-7 and LK-8 had more obvious catalytic advantage compared with LK-2, LK-5, LK-6, and LK-9. Preferably, LK-1, LK-3, LK-4, LK-7 and LK-8 were used for in vitro coupling reaction with IDO.

TABLE 3
Molar yield of α-KG at different
enzymes and substrate concentrations

| | Substrate concentration | | | | |
|---|---|---|---|---|---|
| Enzyme | 50 mM | 100 mM | 150 mM | 200 mM | 300 mM |
| LK | 36.47% | 43.45% | 8.72% | 8.55% | 5.99% |
| LK-1 | 94.48% | 99.24% | 55.35% | 35.10% | 17.87% |
| LK-2 | 6.00% | 36.07% | 4.30% | 0.44% | 2.80% |
| LK-3 | 48.62% | 21.48% | 10.76% | 10.20% | 13.51% |
| LK-4 | 71.28% | 19.47% | 15.96% | 11.23 | 9.32% |
| LK-5 | 4.77% | 6.21% | 2.11% | 4.19% | 1.64% |
| LK-6 | 2.81% | 12.02% | 8.73% | 2.33% | 2.47% |
| LK-7 | 27.36% | 18.10% | 11.45% | 6.44% | 7.03% |
| LK-8 | 28.45% | 22.39% | 11.45% | 7.94% | 4.65% |
| LK-9 | 20.46% | 13.57% | 10.15% | 7.48% | 1.39% |

### Example 5: Synthesis of 4-HILvia a “Two-Step Method” Using Different Self-Assembly Enzyme Systems

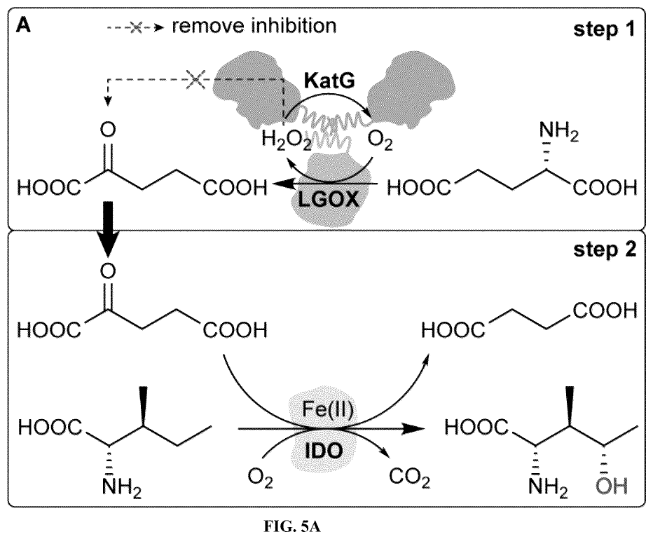

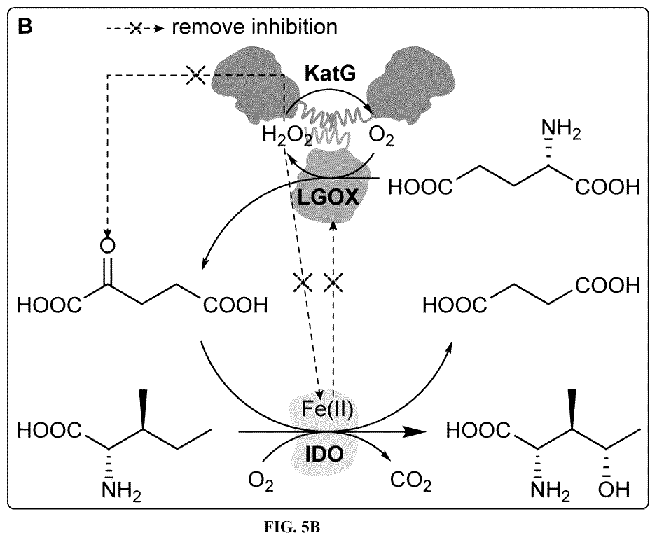

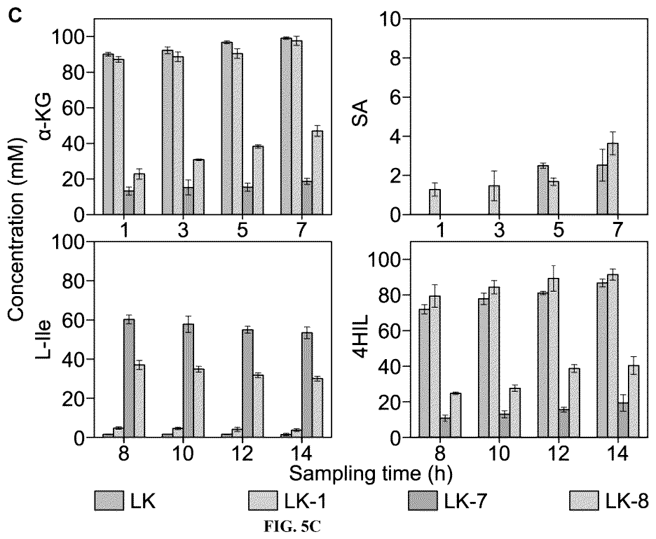

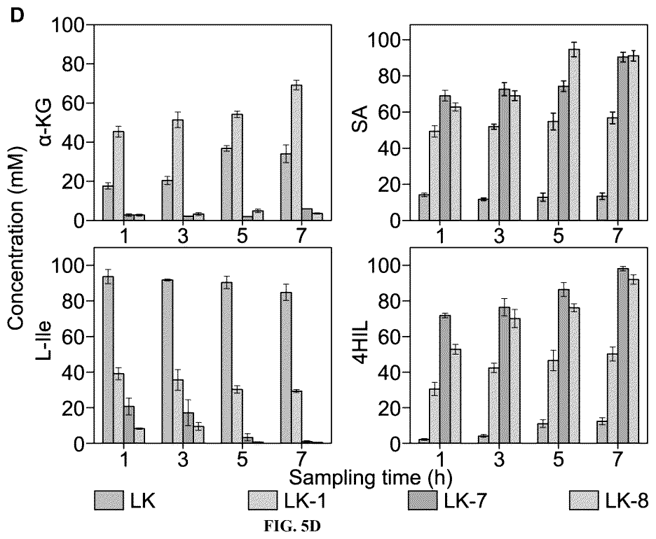

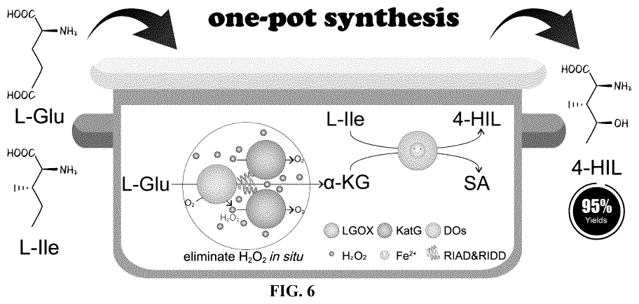

This example illustrates the “two-step method” for synthesizing 4-HIL, involving in vitro L-isoleucine dioxygenase and LGOX-KatG assembled enzyme. The results are shown in FIG. 5 and Table 4.

The L-isoleucine dioxygenase obtained in Example 2 and the assembled enzyme of the multi-enzyme assembly combination screened in Example 4 were used to synthesize 4-HIL by the “two-step method” in vitro, and the mixed solution LK of the free enzyme was used as a control group. The results are shown in Table 4.

The conversion system as divided into two modules and then reacted in two steps: an a-KG generation module and a dioxygenase hydroxylation reaction module, where the α-KG generation module included 100 mM L-Glu, 0.5 g L⁻¹ LGOX (or LGOX-RIAD), and a corresponding two-fold molar concentration of KatG (or KatG-RIDD), 50 mM Tris-HCl buffer (pH 8.0), which were converted at 200 rpm and 30° C. for 7 hours, and then boiled and inactivated for a next-step reaction; and the dioxygenase hydroxylation reaction module was as follows: 100 mM L-Ile, 5 mM FeSO₄, 50 mM Vc, 1 g L⁻¹ IDO, with pH adjusted to 7.0, were added based on the α-KG generation module (providing α-KG as a co-substrate for the hydroxylation reaction), and then converted at 200 rpm and 30° C. for 7 hours.

(1) The L-isoleucine dioxygenase obtained in Example 2 and the free LGOX and KatG obtained in Example 1 were catalyzed at the same concentrations as that of the above-mentioned conversion system. After a two-step biocatalytic conversion for a total of 14 hours, a yield of the target product 4-HIL was 88%, with a space-time yield of 0.92 g L⁻¹ h⁻¹.

(2) The L-isoleucine dioxygenase obtained in Example 2 and the LK-1 screened in Example 4 were subjected to a two-step biocatalytic conversion for a total of 14 hours, resulting in a molar yield of 91.82% for the target product 4-HIL, with a space-time yield of 0.97 g L⁻¹ h⁻¹.

(3) The L-isoleucine dioxygenase obtained in Example 2 and the LK-3 screened in Example 4 were subjected to a two-step biocatalytic conversion for a total of 14 hours, resulting in a molar yield of 77.21% for the target product 4-HIL, with a space-time yield of 0.81 g L⁻¹ h⁻¹.

(4) The L-isoleucine dioxygenase obtained in Example 2 and the LK-4 screened in Example 4 were subjected to a two-step biocatalytic conversion for a total of 14 hours, resulting in a molar yield of 74.41% for the target product 4-HIL, with a space-time yield of 0.78 g L⁻¹ h⁻¹.

(5) The L-isoleucine dioxygenase obtained in Example 2 and the LK-7 screened in Example 4 were subjected to a two-step biocatalytic conversion for a total of 14 hours, resulting in a molar yield of 15.93% for the target product 4-HIL, with a space-time yield of 0.17 g L⁻¹ h⁻¹.

(6) The L-isoleucine dioxygenase obtained in Example 2 and the LK-8 screened in Example 4 were subjected to a two-step biocatalytic conversion for a total of 14 hours, resulting in a molar yield of 37.26% for the target product 4-HIL, with a space-time yield of 0.39 g L⁻¹ h⁻¹.

TABLE 4
Effect of different enzymes on the synthesis
of 4-HILvia the “two-step method”

| Enzyme | Molar yield | Space-time yield |
|---|---|---|
| LK | 88% | 0.92 g L⁻¹ h⁻¹ |
| LK-1 | 91.82% | 0.97 g L⁻¹ h⁻¹ |
| LK-3 | 77.21% | 0.81 g L⁻¹ h⁻¹ |
| LK-4 | 74.41% | 0.78 g L⁻¹ h⁻¹ |
| LK-7 | 15.93% | 0.17 g L⁻¹ h⁻¹ |
| LK-8 | 37.26% | 0.39 g L⁻¹ h⁻¹ |

### Example 6 Synthesis of 4-HILvia a “One-Pot Method” Using Different Self-Assembly Enzyme Systems

This example illustrates the “one-pot method” for synthesizing 4-HIL involving in vitro L-isoleucine dioxygenase and LGOX-KatG assembled enzyme. The results are shown in FIG. 5 and Table 5.

The L-isoleucine dioxygenase obtained in Example 2 and the assembled enzyme of the multi-enzyme assembly combination screened in Example 4 were used to synthesize 4-HIL by the “one-pot method” in vitro, and the mixed solution LK of the free enzyme was used as a control group. A conversion system was 100 mM L-Glu, 0.5 g L⁻¹ LGOX (or LGOX-RIAD) and a corresponding two-fold molar concentration of KatG (or LGOX-RIAD), 100 mM L-Ile, 5 mM FeSO₄, 50 mM Vc and 1 g L⁻¹ IDO, and the conversion was carried out in 50 mM Tris-HCl buffer (pH 7.0) at 200 rpm and 30° C. for 7 hours.

(1) The L-isoleucine dioxygenase obtained in Example 2 and the free LGOX and KatG obtained in Example 1 were catalyzed at the same concentrations as that of the above-mentioned conversion system. After a biocatalytic conversion for a total of 7 hours, a yield of the target product 4-HIL was 4.14%, with a space-time yield of 0.09 g L⁻¹ h⁻¹.

(2) The L-isoleucine dioxygenase obtained in Example 2 and the LK-1 screened in Example 4 were subjected to a biocatalytic conversion for a total of 7 hours, resulting in a molar yield of 50.21% for the target product 4-HIL, with a space-time yield of 1.06 g L⁻¹ h⁻¹.

(3) The L-isoleucine dioxygenase obtained in Example 2 and the LK-3 screened in Example 4 were subjected to a biocatalytic conversion for a total of 7 hours, resulting in a molar yield of 50.69% for the target product 4-HIL, with a space-time yield of 1.07 g L⁻¹h⁻¹.

(4) The L-isoleucine dioxygenase obtained in Example 2 and the LK-4 screened in Example 4 were subjected to a biocatalytic conversion for a total of 7 hours, resulting in a molar yield of 65.25% for the target product 4-HIL, with a space-time yield of 1.38 g L⁻¹ h⁻¹.

(5) The L-isoleucine dioxygenase obtained in Example 2 and the LK-7 screened in Example 4 were subjected to a biocatalytic conversion for a total of 7 hours, resulting in a molar yield of 98.16% for the target product 4-HIL, with a space-time yield of 2.07 g L⁻¹ h⁻¹.

(6) The L-isoleucine dioxygenase obtained in Example 2 and the LK-8 screened in Example 4 were subjected to a biocatalytic conversion for a total of 7 hours, resulting in a molar yield of 92.57% for the target product 4-HIL, with a space-time yield of 1.95 g L⁻¹ h⁻¹.

The LGOX-KatG multi-enzyme self-assembly system mediated by the IAD-RlDD short peptides can efficiently produce ax-KG and simultaneously eliminate the byproduct H₂O₂ produced by LGOX catalysis in situ, thereby efficiently supplying the essential co-substrate α-KG for Fe(II)/ai-KG-dependent dioxygenases in one pot, and promoting a C-H hydroxylation reaction catalyzed by dioxygenases.

TABLE 5
Effect of different enzymes on the synthesis
of 4-HILvia the “one-pot method”

| Enzyme | Molar yield | Space-time yield |
|---|---|---|
| LK | 4.14% | 0.09 g L⁻¹ h⁻¹ |
| LK-1 | 50.21% | 1.06 g L⁻¹ h⁻¹ |
| LK-3 | 50.69% | 1.07 g L⁻¹ h⁻¹ |
| LK-4 | 65.25% | 1.38 g L⁻¹ h⁻¹ |
| LK-7 | 98.16% | 2.07 g L⁻¹ h⁻¹ |
| LK-8 | 92.57% | 1.95 g L⁻¹ h⁻¹ |

### Comparative Example 1

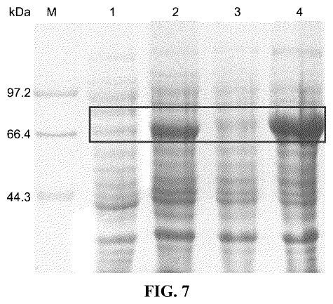

According to the method described in Example 1, the C-terminal of LGOX was linked to the short peptides RIAD and RIDD respectively through a (GGGGS)₃ linker, and the C-terminal of KatG was linked to the short peptides RIAD and RTDD respectively through a (GGGGS)₃ linker, and the crude enzyme activity was measured. The enzymatic activity of resulting crude enzymes was measured. Results are shown in Table 6. SDS-PAGE analysis was also performed, results are shown in FIG. 7.

TABLE 6
Crude enzyme activity

| | Crude enzyme activity |
|---|---|
| Enzyme | (μmol · mg⁻¹ · min⁻¹) |
| LGOX | 30.41 ± 1.84 |
| LGOX-RIAD | 25.5 ± 2.59 |
| LGOX-RIAD | 6.87 ± 1.50 |
| KatG | 286.15 ± 11.65 |
| KatG-RIAD | 318.25 ± 35.25 |
| KatG-RIDD | 323.95 ± 34.35 |

As shown in Table 3 and FIG. 7, LGOX fused with RIDD forms a large amount of inclusion bodies, resulting in a nearly complete loss of enzymatic activity. Therefore, the connection of C-terminal of LGOX with the short peptides RIAD peptide had minimal impact on the enzyme activity.

The above descriptions are merely preferred and feasible embodiments of the present disclosure and are not intended to limit the present disclosure, the present disclosure is not limited to the above examples, and variations, modifications, additions or substitutions made by those skilled in the art within the essential scope of the present disclosure are further within the protective scope of the present disclosure.

What is claimed is:

1. A self-assembly enzyme system, comprising a recombinant protein LGOX-RIAD and a recombinant protein KatG-RIDD; wherein
the recombinant protein LGOX-RIAD is L-glutamate oxidase (LGOX) with a RIAD short peptide added to a C-terminal;
the recombinant protein KatG-RIDD is catalase-peroxidase (KatG) with a RIDD short peptide added to a C-terminal; and
the recombinant protein LGOX-RIAD and the recombinant protein KatG-RIDD are self-assembled through the RIAD short peptide and the RIDD short peptide.

2. The self-assembly enzyme system according to claim 1, wherein a stoichiometric ratio of the recombinant protein LGOX-RIAD to the recombinant protein KatG-RIDD is 1:2.

3. The self-assembly enzyme system according to claim 1, wherein the amino acid sequence of the LGOX is set forth in SEQ ID NO:4.

4. The self-assembly enzyme system according to claim 1, wherein the amino acid sequence of the KatG is set forth in SEQ ID NO:6.

5. The self-assembly enzyme system according to claim 1, wherein the amino acid sequence of the RIAD is set forth in SEQ ID NO:8.

6. The self-assembly enzyme system according to claim 1, wherein the amino acid sequence of the RIDD is set forth in SEQ ID NO:10.

7. The self-assembly enzyme system according to claim 1, wherein the LGOX is linked to the RIAD short peptide using a linker (GGGGS)ₙ, wherein n is 4, 5 or 6; and
the KatG is linked to the RIDD short peptide using a linker (GGGGS)ₙ, wherein n is 4, 5 or 6.

8. The self-assembly enzyme system according to claim 1, wherein the LGOX is linked by (GGGGS)₄ and RIAD to obtain the recombinant protein LGOX-RIAD, and the KatG is linked by (GGGGS)₄ and RIDD to obtain the recombinant protein KatG-RIDD; or
the LGOX is linked by (GGGGS)₆ and RIAD to obtain the recombinant protein LGOX-RIAD, and the KatG is linked by (GGGGS)₄ and RIDD to obtain the recombinant protein KatG-RIDD; or
the LGOX is linked by (GGGGS)₄ and RIAD to obtain the recombinant protein LGOX-RIAD, and the KatG is linked by (GGGGS)₅ and RIDD to obtain the recombinant protein KatG-RIDD; or
the LGOX is linked by (GGGGS)₄ and RIAD to obtain the recombinant protein LGOX-RIAD, and the KatG is linked by (GGGGS)₆ and RIDD to obtain the recombinant protein KatG-RIDD; or
the LGOX is linked by(GGGGS)₅ and RIAD to obtain the recombinant protein LGOX-RIAD, and the KatG is linked by (GGGGS)₆ and RIDD to obtain the recombinant protein KatG-RIDD.

9. A method for multi-enzyme cascade conversion of (2S,3R,4S)-4-hydroxyisoleucine, wherein the method comprises using L-isoleucine and L-glutamic acid as starting substrates, and catalyzing the production of 4-HIL through a cascade reaction using the self-assembly enzyme system in claim 1 and L-isoleucine dioxygenase, employing the self-assembly enzyme system and L-isoleucine dioxygenase cascade catalysis to generate 4-HIL.

10. The method according to claim 9, wherein the multi-enzyme cascade adopts a conversion system for a two-step method or a one-pot method to convert and obtain (2S,3R,4S)-4-hydroxyisoleucine; wherein
the two-step method comprises a first stage and a second stage, wherein in the first stage, monosodium L-glutamate is used as the substrate, and the self-assembly enzyme system is used as a catalyst to prepare and obtain a co-substrate α-ketoglutarate (α-KG); and in the second stage, the co-substrate α-KG obtained in the first stage is used as a starting reaction solution, and L-isoleucine, FeSO₄·7H₂O, L-ascorbic acid, and L-isoleucine dioxygenase are then added to prepare and obtain the (2S,3R,4S)-4-hydroxyisoleucine; and
the one-pot method is to mix monosodium L-glutamate, L-isoleucine, FeSO₄·7H₂O, L-ascorbic acid, L-isoleucine dioxygenase, and the self-assembly enzyme system together to prepare and obtain the (2S,3R,4S)-4-hydroxyisoleucine.

11. The method according to claim 10, wherein for the one-pot method, a concentration of the monosodium L-glutamate is 50-300 mM, a concentration of the L-isoleucine is 50-300 mM, a concentration of the FeSO₄·7H₂O is 1-5 mM, a concentration of the L-ascorbic acid is 10-50 mM, and a concentration of the L-isoleucine dioxygenase is 0.1-1 mg mL⁻¹; and a concentration of the self-assembly enzyme system is 0.1-1 mg mL⁻¹.

12. The method according to claim 10, wherein for the one-pot method, a temperature is 25-35° C., a pH is 7.0-8.0, a conversion lasts for 2-9 hours, and a rotation speed is 200-400 rpm.

13. The method according to claim 10, wherein in the first stage of the two-step method, a concentration of the monosodium L-glutamate is 50-300 mM, and a concentration of the self-assembly enzyme system is 0.1-1 mg mL⁻¹; and in the second stage of the two-step method, a concentration of the L-isoleucine is 50-300 mM, a concentration of the FeSO₄·7H₂O is 1-5 mM, a concentration of the L-ascorbic acid is 10-50 mM, and a concentration of the L-isoleucine dioxygenase is 0.1-1 mg·mL⁻¹.

14. The method according to claim 10, wherein for the two-step method, a temperature is 25-35° C., and a pH is 7.0-8.0; and a conversion in the first stage lasts for 2-9 hours, a conversion in the second stage lasts for 2-9 hours, and a rotation speed is 200-400 rpm.

15. A product, comprising the self-assembly enzyme system of claim 1.

16. A preparation method for the self-assembly enzyme system of claim 1, comprising the following steps:
(1) linking the gene encoding a recombinant protein LGOX-RIAD and the gene encoding a recombinant protein KatG-RIDD into expression vectors to obtain recombinant vectors, respectively; wherein
the recombinant protein LGOX-RIAD is LGOX with a RIAD short peptide added to a C-terminal; and the recombinant protein KatG-RIDD is KatG with a RIDD short peptide added to a C-terminal;
(2) converting the recombinant vectors obtained in the step (1) into Escherichia coli to obtain Escherichia coli expressing LGOX-RIAD and Escherichia coli expressing KatG-RIDD; respectively;
(3) culturing, inducing expression, and purifying the Escherichia coli expressing LGOX-RIAD and the Escherichia coli expressing KatG-RIDD to obtain the recombinant protein LGOX-RIAD and the recombinant protein KatG-RIDD; and
(4) mixing the recombinant protein LGOX-RIAD and the recombinant protein KatG-RIDD to obtain the self-assembly enzyme system.

17. The method according to claim 16, wherein in the step (1), the LGOX is linked to the RIAD short peptide using a linker (GGGGS)ₙ, wherein n is 4, 5 or 6; wherein the amino acid sequence of a basic unit (GGGGS) of the linker is set forth in SEQ ID NO: 12; and
the KatG is linked to the RIDD short peptide using a linker (GGGGS)ₙ, wherein n is 4, 5 or 6; wherein the amino acid sequence of a basic unit (GGGGS) of the linker is set forth in SEQ ID NO:12.

18. The method according to claim 16, wherein the amino acid sequence of the LGOX is set forth in SEQ ID NO:4.

19. The method according to claim 16, wherein the amino acid sequence of the KatG is set forth in SEQ ID NO:6.

20. The method according to claim 16, wherein the amino acid sequence of the RIAD is set forth in SEQ ID NO:8, the amino acid sequence of the RIDD is set forth in SEQ ID NO:10, the expression vectors comprise pET28a, the Escherichia coli is Escherichia coli BL21(DE3); and wherein in the step (4), a stoichiometric ratio of the recombinant protein LGOX-RIAD to the recombinant protein KatG-RIDD is 1:2.
